(19) United States
(12) Patent Application Publication
(10) Pub. No.: US 2019/0082952 A1
(43) Pub. Date: Mar. 21, 2019

(54) APPARATUS AND METHODS OF BLOOD FLOW MEASUREMENT USING OPTICAL COHERENCE TOMOGRAPHY

(71) Applicant: Northwestern University, Evanston, IL (US)

(72) Inventors: Hao F. Zhang, Deerfield, IL (US); Xiao Shu, Evanston, IL (US); Wenzhong Liu, Evanston, IL (US); Lian Duan, Evanston, IL (US)

(21) Appl. No.: 16/123,518

(22) Filed: Sep. 6, 2018

Related U.S. Application Data
(60) Provisional application No. 62/554,724, filed on Sep. 6, 2017.

(51) Int. Cl. A61B 3/10; A61B 5/00; A61B 3/12

(52) U.S. Cl. CPC A61B 3/102; A61B 5/0073; A61B 5/02007; A61B 3/1241; A61B 5/0066; A61B 5/7257

(57) ABSTRACT

Systems and methods to measure blood flow using optical coherence tomography are disclosed. An example method includes: performing scanning of a target with beam(s) of incident low coherence radiation, wherein the low coherence radiation is selected from one or more regions of the electromagnetic spectrum based on the target selected; acquiring spectroscopic information from scanning signals generated by the interaction of the incident low coherence radiation and the target; generating image signal data for the target from the scanning signals; processing the image signal data by selecting electro-magnetic property(-ies) related to the modulation of the low coherence radiation by variation of relative permittivity and conductivity of the target; performing a multivariate comparison of the selected electro-magnetic properties related to the modulation of the low coherence radiation by variation of relative permittivity and conductivity of the target; and quantifying motion property(-ies) of the target.

### CROSS-REFERENCE TO RELATED APPLICATIONS

This patent claims the benefit of U.S. Provisional Application Ser. No. 62/554,724, entitled “Method of Blood Flow Measurement Using Optical Coherence Tomography,” which was filed on Sep. 6, 2017, and which is hereby incorporated herein by reference in its entirety for all purposes.

### STATEMENT REGARDING FEDERALLY SPONSORED RESEARCH FOR DEVELOPMENT

This invention was made with government support under DP3DK108248 and R01EY026078 awarded by the National Institutes of Health. The government has certain rights in the invention.

### BACKGROUND OF THE DISCLOSURE

Optical coherence tomography (OCT) is a non-invasive optical imaging technique which produces depth-resolved reflectance imaging of samples through the use of a low coherence interferometer system. OCT imaging allows for three-dimensional (3D) visualization of structures in a variety of biological systems and non-biological systems not easily accessible through other imaging techniques. In some instances, OCT may provide a non-invasive, non-contact approach to assess information without disturbing or injuring a target or sample. In some examples, function optical coherence tomography (fOCT) can provide additional information regarding physical and chemical attributes inside vessels and structures, such as measurements of fluid flow. In medical applications, fOCT measurements can be used for diagnostic or monitoring purposes of a variety of fluids in the treatment of various diseases.

In ophthalmic applications, previous experimental investigations have focused on the measurement of retinal blood flow rate with various types of OCT, such as spectral domain optical coherence tomography (SD-OCT), for the diagnosis and monitoring of various ocular diseases. In some examples, to measure blood flow, both the vessel size and the blood velocity must be quantified. For example, using SD-OCT, retinal vessel size is normally extracted from a cross-sectional OCT B-scan image, due to circumstances in which axial resolution may be higher than lateral resolution with this type of OCT. Several techniques have been proposed to extract retinal blood flow velocity: (1) the indirect method which employs first calculating the phase shift variance or light intensity within vessels, and then calibrating the measured results using well-controlled phantoms; and (2) the mean phase shift method, in which the phase shift between two adjacent A-lines can be used to directly quantify axial flow velocity. However, this latter method requires the Doppler angle (i.e., the angle between the probing beam and retinal vessels) to measure absolute velocity.

For either method, the Doppler angle must be either measured or circumvented in calculating fluid flow velocity. While methods exist to solve the issue of the Doppler angle in these calculations, such as the multiple-beams scanning scheme, or the en face Doppler approach, such methods require the objective focal length between the objective and the target. In ophthalmic applications, where an eyeball is scanned using OCT, this objective focal length may include the axial length of the eyeball, wherein the retina is the target or subject. The multiple-beams scanning approach requires the eyeball axial length in order to access the geometrical information of the retinal vessels, which thus enables the Doppler angles calculation. The en face Doppler method requires the eyeball axial length to calibrate a transverse scanning dimension to measure flow rate.

For ophthalmic applications, instead of measuring the eyeball axial length for every subject, a more commonly applied approach may be use of a universal empirical eyeball axial length for flow quantification. Commonly this distance is fixed as an assumption in calculations of fluid flow velocity. In reality however, eyeball axial length may vary with age (e.g., the aged eye may have a significantly shorter axial length), vary with other eye conditions or vary due to anatomical variance between individuals. In patients with myopia, for instance, the eyeball axial length of subjects with −4.36 D nearsightedness can be 5.2 mm shorter than subjects with −6.00 D. The use of a universally assumed path length, as in the case of assuming a universal eyeball axial length for all patients or subjects, can affect the accuracy the flow velocity determination. There is need in the art to improve the accuracy of quantifying fluid flow using fOCT, where the objective focal length is unknown or no empirical measurement is possible, such as in measuring retinal blood flow in the eye without knowing or previously measuring the axial length of the eyeball.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of methods and systems of this disclosure are set forth with particularity in the appended claims. A better understanding of the features and advantages of this disclosure will be obtained by reference to the following detailed description that sets forth illustrative examples, in which the principles of the methods and systems of this disclosure are utilized, and the accompanying drawings.

FIG. 1(a) depicts a selection of Gaussian windows for sub-bands of the OCT spectrum.

FIG. 1(b) shows example sub-band dependent Doppler phase shifts under different axial displacements.

FIG. 2 illustrates a flow diagram of an example process of spectroscopic Doppler analysis.

FIGS. 3(a)-3(c) illustrate schematics of an example spectroscopic Doppler analysis system for vis-OCT blood flow velocity measurement.

FIG. 4(a) shows example averaged Doppler phase shift images of two selected flow velocities before and after phase unwrapping.

FIG. 4(b) shows example sub-band dependent phase shifts using spectroscopic Doppler analysis.

FIG. 5(a) shows example sub-band dependent phase shifts for seven flow rates.

FIG. 5(b) shows example measured flow rates using conventional and spectroscopic Doppler analysis.

FIG. 6 shows example axial retinal blood flow velocities measured by conventional and spectroscopic Doppler analyses, both converted to radian values for comparison.

FIGS. 7-9 illustrate example computer systems to implement the apparatus, systems, and methods disclosed herein.

FIG. 10 illustrates an example OCT data processing system.

FIG. 11 illustrates an example flow diagram of a method to evaluate a target.

The following detailed description of certain examples of the present invention will be better understood when read in conjunction with the appended drawings. For the purpose of illustrating the invention, certain examples are shown in the drawings. It should be understood, however, that the present invention is not limited to the arrangements and instrumentality shown in the attached drawings.

### DETAILED DESCRIPTION OF THE DISCLOSURE

### I. General Overview

The methods and systems of the present disclosure provide for the determination of fluid flow using fOCT. Generally, determination of fluid flow and fluid flow rates using fOCT may include a non-invasive, non-contact method for determining a functional state of target, such as the health of bodily tissue. In some examples, fOCT objective focal length free measurements may be used for determining the change in metabolism of a tissue, therefore indicating something about disease state or health.

Generally, fOCT employs any method of OCT, as known in the art. fOCT objective focal length free measurements provides a method of determining flow rates of fluid without knowing or measuring the objective focal length. As previously described, such as in U.S. Pat. Nos. 9,046,339 and 8,244,334, the methods of which are incorporated by reference herein, determining fluid flow using OCT often requires measurement of the objective focal length before OCT scanning, or an assumed universal length is used for fluid flow calculations. Measurements of the objective focal length, in some examples the axial length of an eyeball for ophthalmic applications, can be cumbersome or impossible. A universal assumption of the objective focal length, such as a universal assumption of the axial length of the eye can also lead to inaccurate results. Certain examples can determine fluid flow using OCT without need for objective focal length measurement, predetermination or assumption of length.

### II. General Methods for Flow Measurement

The terms “optical coherence tomography” and “OCT,” described herein, generally refer to an interferometric technique for imaging samples, in some examples, with micrometer lateral resolution. This non-invasive optical tomographic imaging technique is used in a variety of medical and industrial applications to provide cross-sectional or 3D images of a target.

The terms “functional OCT” and “fOCT,” described herein, generally refer to a method of OCT imaging that provides for the acquisition of both structural (3D, tomographic and cross-sectional information) and functional information about a target, as described herein. In some examples, fOCT may refer to “visible-OCT” or “vis-OCT.” Vis-OCT generally refers to a type of fOCT that includes use of visible light. In some examples, OCT or fOCT may refer to OCT methods comprising use of near infrared (NIR) light.

As describe herein, fOCT may utilize any method of OCT. Generally, fOCT may be configured with an interferometer, as is the case for many other OCT methods. Light from a light source (for example, a broadband light source) is split (for example, by a beam-splitter) and travels along a sample arm (generally comprising the sample) and a reference arm (generally comprising a mirror). A portion of the light from the sample arm illuminates a target. Light is also reflected from a mirror in the reference arm. (Light from the test arm and the reference arm is recombined, for example, by the beam-splitter.) When the distance travelled by light in the sample arm is within a coherence length of the distance travelled by light in the reference arm, optical interference occurs, which affects the intensity of the recombined light. The intensity of the combined reflected light varies depending on the target properties. Thus, variations for the intensity of the reflectance measured are indications of the physical features or attributes of the target being imaged. Configuration of the system can vary as described further below.

In some examples, the methods and systems of the disclosure may utilize time-domain OCT, where the length of the reference arm can be varied (for example, by moving one or more reference mirrors). The reflectance observed as the reference arm distance changes indicates sample properties at different depths of the sample. In some examples, the length of the sample arm is varied instead of or in addition to the variation of the reference arm length. In some examples, the devices, methods and systems may utilize frequency-domain OCT, where the distance of the reference arm can be fixed, and the reflectance can then be measured at different frequencies. For example, the frequency of light emitted from a light source can be scanned across a range of frequencies or a dispersive element, such as a grating, and a detector array may be used to separate and detect different wavelengths. Fourier analysis can convert the frequency-dependent reflectance properties to distance-dependent reflectance properties, thereby indicating sample properties at different sample depths. In certain examples, OCT can show additional information or data not obtainable from other forms of imaging.

In some examples, the methods and systems of the disclosure may utilize frequency-domain optical coherence tomography, where the reference and sample arms are fixed. Light from a broadband light source comprising a plurality of wavelengths is reflected from the sample and interfered with light reflected by the reference mirror/s. The optical spectrum of the reflected signal can be obtained. For example, the light may be input to a spectrometer or a spectrograph, comprising, for example, a grating and a detector array that detects the intensity of light at different frequencies.

In some examples, the methods and systems of the disclosure may utilize spectral domain optical coherence tomography, whereby spectral information is extracted by distributing different optical frequencies onto a detector stripe (for example, a line-array CCD or CMOS) via a dispersive element. Information of the full depth scan can be acquired within a single exposure.

Fourier analysis may be performed, for example, by a processor, and may convert data corresponding to a plurality of frequencies to that corresponding to a plurality of positions within the sample. Thus, data from a plurality of sample depths can be simultaneously collected without the need for scanning of the reference arm (or sample) arms. Additional details related to frequency domain optical coherence tomography are described in Vakhtin et al., (Vakhtin A B, Kane D J, Wood W R and Peterson K A. “Common-path interferometer for frequency-domain optical coherence tomography,” Applied Optics. 42(34), 6953-6958 (2003)) and incorporated by reference herein.

Other methods of performing optical coherence tomography are possible. For example, in some cases of frequency domain optical coherence tomography, the frequency of light emitted from a light source varies in time. Thus, differences in light intensity as a function of time relate to different light frequencies. When a spectrally time-varying light source is used, a detector may detect light intensity as a function of time to obtain optical spectrum of the interference signal. The Fourier transform of the optical spectrum may be employed as described herein. The devices, methods and systems of the disclosure may utilize any method of OCT, including but not limited to spectral domain OCT, Fourier domain OCT, time encoded frequency domain OCT, or swept source OCT, single point OCT, confocal OCT, parallel OCT, or full field OCT as known in the art.

Generally, the term “A-scan” OR “A-line” describes the light reflectivity associated with different sample depths. The term “B-scan” or “B-line” as used herein refers to the use of cross-sectional views of tissues formed by assembly of a plurality of A-scans. In the case of fOCT methods of cancer detection, light reflected by cancerous tissue target is converted into electrical signals and can be used to generate both cross-sectional or 3D structural images and metabolic functional information about the target tissue (such as cancerous growth, lesion, or tumor). In the case of ophthalmology, light reflected by eye tissues is converted into electrical signals and can be used to provide data regarding the 3D structure of tissue in the eye and metabolic activity in the retina. In many cases, including but not limited to cancer detection and ophthalmology, A-scans and B-scans can be used, for example, for differentiating normal and abnormal tissue.

For general methods, an A-scan can generally include collecting data at one or more transverse locations in a target, at a plurality of depths in a z-axis direction; a B-scan may include cross-sectional data from a medial border to a lateral border, or (x,y) axis direction. In the case of fOCT of a skin cancer lesion for example, an A-scan can generally include data from the outer regions of the epidermis of the lesion to the inner regions comprising vasculature, while B-scans can include cross sectional data from one lesion border to another in the (x,y) plane. In ophthalmic instances, an A-scan can generally include data from the cornea to the retina, and a B-scan can include cross-sectional data from a medial border to a lateral border of the eye and from the cornea to the retina. 3D C-scans may be used to generate one or more 3D images by combining a plurality of B-scans in variety of examples.

In the present disclosure, “target” may indicate any sample, object, or subject suitable for imaging. In some examples, a target may include but is not limited to inanimate material such as metals, alloys, polymers, and minerals as found for industrial applications for fOCT and as described herein. In some examples, a target may be animate material, such any suitable living material including but not limited to embryos, seeds, cells, tissues, grafts, blood vessels, organs, or organisms as would be suitable for medical and agricultural applications for fOCT as described herein. In some examples, a target may be retinal tissue, etc.

In the present disclosure, objective focal length may refer to any distance or length between the OCT objective and the target. For example, an OCT device or system may generate beams of radiation on a target. The objective focal length may refer to a distance between the OCT device and the target. In some examples, the target may contain one or more vessels or blood vessels, through which fluid flows at some velocity. The general methods and system of the present disclosure provide for the determination of fluid flow without relying on predetermining, measuring, or assuming the objective focal length.

In some examples, where the target is the retina of an eyeball, the objective focal length may be the axial length of an eyeball. Light beams from an fOCT instrument enter the eyeball through the outside of the cornea at V and hit the retina at F′. In some examples, the axial length of the eye may be considered the distance from V to F′, or the sum of lengths of endpoints V and H′ and endpoints H and F′, or l′. In some examples, axial length may refer to the length l′, the distance between endpoints H′ and F′.

In some cases, axial fluid flow components may refer to physical parameters relating to the movement of one or more particles in the fluid. For example, in blood, one or more blood components, such as blood cells may be imaged by Doppler OCT. axial fluid components of individual red blood cells in a blood vessel may include but are not limited to the blood vessel diameter, the velocity of the red blood cell and the Doppler angle of the imaging beam of radiation, as described herein.

The methods and systems of the present disclosure may use any light source suitable for OCT, including but not limited to supercontinuum lasers, superluminescent diodes, continuous wave lasers or ultrashort pulsed lasers. The light source may be used to generate one or more low coherence beams of radiation or light to illuminate the target, for example.

The calculation methods described herein may be performed by a software algorithm or computer of the OCT device/system. Generally, OCT scanning data is acquired by the OCT device or system and subsequently analyzed through the calculation methods described herein. The absolute flow rate F of the target can be expressed as any unit of distance divided by a time unit. In some examples, where the target sample is one or more retinal vessels in an eye, the absolute flow rate may be expressed as μl/min. Generally, axial flow components are a combination of absolute flow velocity V, which can be expressed as any suitable units of distance divied by time, (e.g. mm/s), and the perpendicular cross-sectional vessel size S of the vessel, (e.g. μm²). In some examples, the absolute flow rate F can be determined by multiplying the absolute flow velocity V by the perpendicular cross-sectional vessel size S of the vessel. Alternatively, F can also be quantified by the detected mean projected velocity V. and the measured vessel area Sₘ from Doppler OCT.

A target may include any vessel or structure that can contain a fluid to be imaged including but not limited to tissue, healthy tissue, diseased tissue, retina, tumor, cancer, growth, fibroid, lesion, skin, mucosal lining, organ, graft, blood supply and one or more blood vessels.

In some examples, a fluid may be any material capable of flow, in which there may be particles that may be imaged by OCT or Doppler OCT. Bodily fluid may include but is not limited to whole blood, blood plasma, blood serum, urine, semen, tears, sweat, saliva, lymph fluid, pleural effusion, peritoneal fluid, meningal fluid, amniotic fluid, glandular fluid, spinal fluid, conjunctival fluid, vitreous, aqueous, vaginal fluid, bile, mucus, sputum and cerebrospinal fluid.

In some examples, target function may include but is not limited to metabolic activity, metabolic rate, oxygen consumption, tissue consumption of a biomarker or analyte, pathophysiological alterations, pathological alterations, histological change such as tissue remodeling, abnormal growth of one or more blood vessels, or abnormal tissue growth, necrosis, apoptosis, necrosis, angiogenesis, cell proliferation, neurmodulation, neural activity, wound healing, infection, burns, scarring, radiological damage, hypoxia, oxidative stress and the like.

In some examples, measurements regarding flow rate of fluid such as blood may be used to compute or determine target function. For example, measurements regarding the flow rate of blood may help determine the flow rate of oxygen (via hemoglobin transport) into or out of a particular target or region. The flow of oxygen may be a critical factor in determining metabolic activity, histological change such as tissue remodeling, abnormal growth of one or more blood vessels, or abnormal tissue growth, necrosis, apoptosis, necrosis, angiogenesis. In other examples, the measurements of flow of other analytes or cells in fluids such as cerebrospinal fluid (CSF), may indicate the presence of disease of infection or inflammation of one or more parts of the nervous system.

In some example, a change in target function may be determined by comparing information from flow measurement of a fluid to a reference. In some examples, a reference many include but is not limited to measurements of from a healthy or normal target, one or more previous measurements, or an average of information from healthy subjects. In some examples, a reference may include flow measurement at different times. In some examples, one or more references may be compared to other references to determine a change in flow measurements.

In various examples, one or more fOCT images may provide flow measurement data from which a diagnosis and/or evaluation may be made. In some examples, such determinations may relate to biologic tissue structure, vasculature, and/or microcirculation. In some examples, measurements of blood flow through individual vessels therein may be useful in understanding mechanisms behind a number of disease developments and treatments including, for example, ischemia, degeneration, trauma, seizures, and various other neurological diseases. In still other examples, an fOCT flow measurement data image and techniques herein disclosed may be used to identify cancer, tumors, dementia, and ophthalmologic diseases/conditions (including, e.g., glaucoma, diabetic retinopathy, age-related macular degeneration). Still further, in various examples, OCT techniques as herein disclosed may be used for endoscopic imaging or other internal medicine applications. In some examples, fOCT flow measurement data may be used to stratify treatment options, such as personalizing or tailoring a patient treatment specific treatment protocol. The foregoing examples of diagnosis and/or evaluation are provided for purposes of illustration only, and, thus, embodiments of the present invention are not limited to the examples disclosed and described herein.

fOCT flow measurement data may be used in medical decisions related to a variety of diseases. These may include neurological diseases, which may include but is not limited to dementia, concussion, Alzheimer's disease, Parkinson's disease, peripheral neuropathy, epilepsy and multiple sclerosis. In some examples, these may include vascular diseases, including but not limited to diabetes, peripheral vascular diseases, stroke, cardiovascular diseases, myocardial infarction, and aneurysm.

In some examples, fOCT flow measurement data may be used to provide medical decision for ocular diseases which may include but is not limited to autosomal retinitis pigmentosa, autosomal dominant retinitis punctual albescens, butterfly-shaped pigment dystrophy of the fovea, adult vitelliform macular dystrophy, Norrie's disease, blue cone monochromasy, choroideremia, gyrate atrophy, age-related macular degeneration, retinoblastoma, anterior and posterior uveitis, retinovascular diseases, cataracts, corneal dystrophies, retinal detachment, degeneration and atrophy of the iris, and diabetic retinopathy, herpes simplex virus infection, cytomegalovirus, allergic conjunctivitis, dry eye, lysosomal storage diseases, glycogen storage diseases, disorders of collagen, disorders of glycosaminoglycans and proteoglycans, sphinogolipodoses, mucolipidoses, disorders of amino acid metabolism, dysthyroid eye diseases, anterior and posterior corneal dystrophies, retinal photoreceptor disorders, corneal ulceration, glaucoma and ocular wounds.

In some examples, fOCT flow measurement data may be used for medical decisions related to cancer, for example, acute myeloid leukemia; bladder cancer, including upper tract tumors and urothelial carcinoma of the prostate; bone cancer, including chondrosarcoma, Ewing's sarcoma, and osteosarcoma; breast cancer, including noninvasive, invasive, phyllodes tumor, Paget's disease, and breast cancer during pregnancy; central nervous system cancers, adult low-grade infiltrative supratentorial astrocytoma/oligodendroglioma, adult intracranial ependymoma, anaplastic astrocytoma/anaplastic oligodendroglioma/glioblastoma multiforme, limited (1-3) metastatic lesions, multiple (>3) metastatic lesions, carcinomatous lymphomatous meningitis, nonimmunosuppressed primary CNS lymphoma, and metastatic spine tumors; cervical cancer; chronic myelogenous leukemia (CIVIL); colon cancer, rectal cancer, anal carcinoma; esophageal cancer; gastric (stomach) cancer; head and neck cancers, including ethmoid sinus tumors, maxillary sinus tumors, salivary gland tumors, cancer of the lip, cancer of the oral cavity, cancer of the oropharynx, cancer of the hypopharynx, occult primary, cancer of the glottic larynx, cancer of the supraglottic larynx, cancer of the nasopharynx, and advanced head and neck cancer; hepatobiliary cancers, including hepatocellular carcinoma, gallbladder cancer, intrahepatic cholangiocarcinoma, and extrahepatic cholangiocarcinoma; Hodgkin disease/lymphoma; kidney cancer; melanoma; multiple myeloma, systemic light chain amyloidosis, Waldenstrom's macroglobulinemia; myelodysplastic syndromes; neuroendocrine tumors, including multiple endocrine neoplasia, type 1, multiple endocrine neoplasia, type 2, carcinoid tumors, islet cell tumors, pheochromocytoma, poorly differentiated/small cell/atypical lung carcinoids; Non-Hodgkin's Lymphomas, including chronic lymphocytic leukemia/small lymphocytic lymphoma, follicular lymphoma, marginal zone lymphoma, mantle cell lymphoma, diffuse large B-Cell lymphoma, Burkitt's lymphoma, lymphoblastic lymphoma, AIDS-Related B-Cell lymphoma, peripheral T-Cell lymphoma, and mycosis fungoides/Sézary Syndrome; non-melanoma skin cancers, including basal and squamous cell skin cancers, dermatofibrosarcoma protuberans, Merkel cell carcinoma; non-small cell lung cancer (NSCLC), including thymic malignancies; occult primary; ovarian cancer, including epithelial ovarian cancer, borderline epithelial ovarian cancer (Low Malignant Potential), and less common ovarian histologies; pancreatic adenocarcinoma; prostate cancer; small cell lung cancer and lung neuroendocrine tumors; soft tissue sarcoma, including soft-tissue extremity, retroperitoneal, intra-abdominal sarcoma, and desmoid; testicular cancer; thymic malignancies, including thyroid carcinoma, nodule evaluation, papillary carcinoma, follicular carcinoma, Hürthle cell neoplasm, medullary carcinoma, and anaplastic carcinoma; uterine neoplasms, including endometrial cancer and uterine sarcoma.

In some examples, a medical decision may be facilitated by comparing the bodily fluid flow in a target (e.g. tissue), by comparing the bodily fluid flow in the target to the bodily fluid flow in a target control. In some cases, the differences in body fluid flow of a target and bodily fluid flow of target control may be indicative of disease. For example, in some cases, bodily fluid flow may be obstructed or reduced by a disease state, such as abnormal clotting, or blood vessel leakage, wherein the bodily fluid is blood. In some cases, a target control may be a healthy target. In some cases, a target control may be healthy tissue. In some cases, a target control may be a target that is known to be or suspected to be free of disease. In some cases, a target and target control may be in the same person, tissue or subject. In some cases, a target and target control may be in a different person, tissue or subject. In some cases, disease detection or stratification of treatment options may be performed wherein the bodily fluid flow is reduced as compared to a target control. In some cases, a disease detection or stratification of treatment options may be performed wherein the bodily fluid flow is increased as compared to a target control.

### III. Example Systems and Methods of Spectroscopic Doppler Analysis for Visible-Light OCT

Retinal oxygen metabolic rate can be effectively measured by visible-light optical coherence tomography (vis-OCT), which simultaneously quantifies oxygen saturation and blood flow rate in retinal vessels through spectroscopic analysis and Doppler measurement, respectively. Doppler OCT relates phase variation between sequential A-lines to the axial flow velocity of the scattering medium. The detectable phase shift is between −π and π due to its periodicity, which limits the maximum measurable unambiguous velocity without phase unwrapping. Using shorter wavelengths, vis-OCT is more vulnerable to phase ambiguity since flow induced phase variation is linearly related to the center wavenumber of the probing light. In certain examples, phase unwrapping can be eliminated using spectroscopic Doppler analysis. For example, the vis-OCT spectrum can be separated into a series of narrow sub-bands, and vis-OCT images can be reconstructed to extract corresponding Doppler phase shifts in all the sub-bands. Then, flow velocity can be quantified by analyzing sub-band dependent phase shift using linear regression. Spectroscopic Doppler analysis extends a measurable absolute phase shift range without conducting phase unwrapping.

Retinal metabolic rate of oxygen (rMRO2) is a parameter critical for the fundamental investigation and clinical diagnosis of several blinding diseases such as age-related macular degeneration, glaucoma and, most importantly, diabetic retinopathy (DR). Previous studies suggested that retinal metabolic abnormalities play an important role in the development of DR and that rMRO2 is a potential biomarker for early diagnosis of DR before irreversible damages occur. To calculate rMRO2, oxygen saturation (sO2) is to be measured along with blood flow rate in major retinal blood vessels, both of which can be effectively quantified simultaneously by visible-light optical coherence tomography (vis-OCT). As a single imaging modality for rMRO2 measurement, vis-OCT detects sO2 by retrieving absorption spectrum of blood from spectroscopic analysis and measures flow rate using a Doppler OCT method. Compared with conventional OCT using near infrared (NIR) light, vis-OCT provides reliable sO2 measurement, higher resolution, but has reduced measurement range of flow velocity.

The reduced flow measurement range of vis-OCT originates from the underlying mechanism of Doppler OCT, which relates phase shift between sequentially acquired OCT A-lines to the axial displacement of scatterers. The axial displacement is converted to flow velocity based on the A-line rate of system and Doppler angle, which is the angle between flow direction and light probing axis. Since phase variation is periodic, the actual detectable Doppler phase shift always lies between −π and π. Phase wrapping occurs when flow induced axial displacement exceeds the center wavelength of the probing beam. Therefore, using shorter wavelengths, vis-OCT is more sensitive to low flow velocity but more susceptible to phase wrapping in measuring high flow velocity.

The measurable velocity range of vis-OCT can be extended in a few ways. First, phase unwrapping can be applied to increase the range beyond −π and π. However, although various unwrapping techniques have been investigated, it remains challenging to remove artifacts induced by phase wrapping in clinical settings. Second, the phase shift induced by a certain flow velocity can be reduced by decreasing the time interval between two sequential observations, which is the inverse of A-line acquisition rate. This can be done by decreasing spectrometer integration time of spectral-domain OCT (SD-OCT) or by switching to a fast swept-source system. However, a reduced spectrometer integration time leads to a lower signal-to-noise ratio (SNR) and a swept-source for visible light is not yet available. Third, the imaging operator can intentionally position the OCT probing beam and retinal blood vessel for a larger Doppler angle to decrease the projected axial velocity. However, this procedure can significantly increase the operation complexity and may be impractical for the region around optic nerve head.

Certain examples extend flow velocity measurement range for vis-OCT without using sophisticated phase unwrapping. In most existing Doppler OCT algorithms, the center wavelength or wavenumber of the broadband light source is used to translate the detected phase shift to the axial displacement of scatters. However, if the broad spectrum is split into a number of narrower sub-bands for OCT reconstruction, a series of Doppler phase shifts can be obtained, and these phase shifts follow a linear relationship with the center wavenumbers of the sub-bands. A slope of this linear function, rather than actual phase shift values, can be used to retrieve the axial flow velocity of the scatterers, which is independent of phase wrapping. Thus, spectroscopic Doppler analysis can extend the maximum blood flow measurement limit beyond −π and π, which is especially beneficial in vis-OCT.

Certain examples provide systems and methods for spectroscopic Doppler analysis to quantify flow velocity in spectral-domain vis-OCT. In certain examples, a series of short-time Fourier transforms (STFT) of the original broadband OCT interferogram are performed and Doppler phase shifts are obtained from different sub-bands. Linear regression can be applied to wavenumber-dependent phase shift values, and axial flow velocity can be retrieved from the fitted slope. The algorithm is validated using a vis-OCT system with a 100-nm bandwidth at a center wavelength of 560 nm, for example. Phantom experiments show that both conventional Doppler and the spectroscopic Doppler analysis methods measure flow velocities accurately when the velocity was low, but spectroscopic Doppler analysis increases the measurable range of the flow velocity. Spectroscopic Doppler analysis can be applied to measure in vivo retinal blood flow, for example.

Doppler OCT Examples

SD-OCT measures flow velocity by detecting phase difference between two sequential A-lines. If a single moving scatterer appears at depths of z0 and z0+δz in the first and second A-lines, corresponding interferograms i(k) of the two flow velocity measurements can be described as:

i₀(k)=ξs(k)\sqrt{R_{R}R_{S}} cos(2nkz₀) (Eq. 1), and

i₁(k)=ξs(k)\sqrt{R_{R}R_{S}} cos[(2nk(z₀+δz)] (Eq. 2),

where ξ is a constant determined by optical detector quantum efficiency; s(k) is a spectral power density function of a light source; R_{R} and R_{S} are reflectivities of a reference mirror and a sample scatterer, respectively; n is a refractive index; and k is an angular wavenumber, defined as 2π/λ (λ is the wavelength). Applying a Fourier transform to i₀(k) and i₁(k), complex OCT A-lines I₀(z) and I₁(z) can be obtained. If δz is much smaller than OCT axial resolution, a modulus of the complex A-line, corresponding to an OCT structure image, is insensitive to scatter displacement. However, a phase shift term can be added to I₁(z), which is proportional to δz:

I₀(z)=ξ\sqrt{R_{R}R_{S}}[S(z−2nz₀)+S(z+2nz₀)] (Eq. 3),

I₁(z)=ξ\sqrt{R_{R}R_{S}}[S(z−2nz₀)e^{j2nk}^{_{equ}}^{δz}+S(z+2nz₀)e^{-j2nk}^{_{equ}}^{δz}] (Eq. 4),

Δφ_{D}=2nk_{equ}δz (Eq. 5),

where S(z) is an autocorrelation of s(k); k_{equ} is an equivalent wavenumber of the light source, which can be calculated as:

$${k}_{equ} = \frac{\sum k \cdot s ( k )}{\sum s ( k )} . \qquad ( Eq . 6 )$$

For a light source with symmetric spectrum, center wavenumbers k_{c} and k_{equ} are interchangeable in Eq. (6). However, when a source spectrum is asymmetric, using k_{c} directly leads to a bias in flow velocity measurement.

An acquisition time interval between two sequential A-lines can be represented as T, which can be determined by a spectrometer integration time in SD-OCT or a wavelength sweeping cycle in a swept-source OCT (SS-OCT). Eq. (5) can be rearranged to obtain an axially projected flow velocity v_{∥} by taking a ratio of the axial displacement δz and T as follows:

$${v}_{\vert \vert} = \frac{{Δϕ}_{D}}{2 {nTk}_{equ}} . \qquad ( Eq . 7 )$$

Phase wrapping occurs when the axial displacement-induced absolute phase shift exceeds π, which results in ambiguous axial flow velocity measurement. The phase wrapping limited maximum absolute axial velocity (PLV) is given by:

$${v}_{\vert \vert , wrap} = \frac{π}{2 {nTk}_{equ}} . \qquad ( Eq . 8 )$$

Another factor that limits the flow detection is a washout effect, which describes a signal loss due to averaging of motion-varied interferometric fringes over an integration time of the optical detector. For SD-OCT, washout effect starts at around PLV and causes a Doppler phase signal to be unrecoverable beyond twice PLV.

Spectroscoptic Doppler OCT

STFT has been used in spectroscopic analysis of OCT signals to extract wavenumber-dependent absorption and scattering properties in tissue, which can be described as:

STFT{i(k)}(τ,ω)=DFT[i(k)w(k,τ,ω)] (Eq. 9),

where τ and ω are a center wavenumber and bandwidth of each OCT sub-band, respectively; DFT is a discrete Fourier transform; and w(·) represents a Gaussian window function. Since the Gaussian window gives more weight to the central wavelength in the sub-band, the equivalent wavenumber defined in Eq. (6) can be estimated by the center wavenumber τ. If the axial flow velocity is v∥, then a detected Doppler phase shift in each sub-band can be calculated from Eq. (7) as:

Δφ_{D}(τ)=2nTv_{∥}τ (Eq. 10),

which shows that a series of Δφ_{D}(τ) can be obtained by sliding a window function across a broadband OCT spectrum, and that the Doppler phase shift is a linear function of the center wavenumber with a slope of the linear function proportional to the axial flow velocity. If a is used to represent the slope of the linear function, the axial flow velocity can be calculated as:

$${v}_{\vert \vert} = \frac{a}{2 nT} . \qquad ( Eq . 11 )$$

Spectroscopic analysis and linear fitting can be used to determine slope a and axial flow velocity v∥.

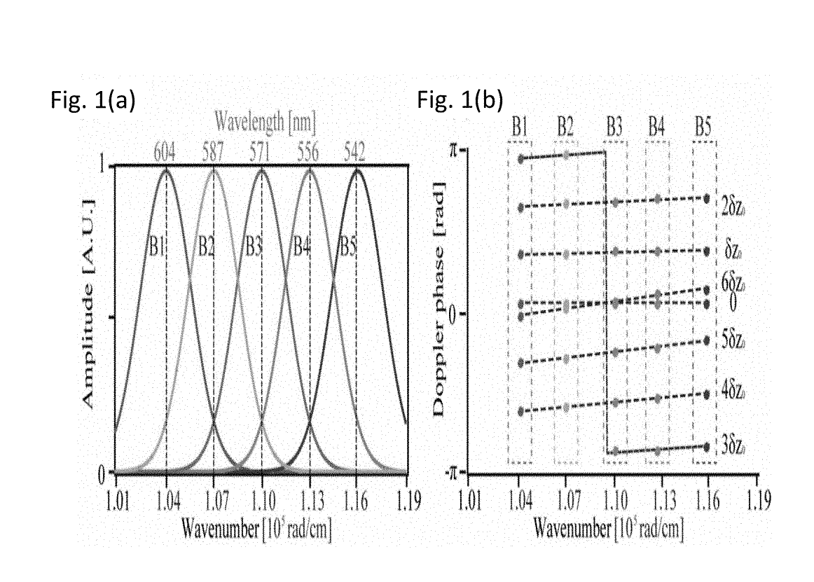

In certain examples, Doppler phase shift varies with respect to center wavenumber of OCT sub-band. Though OCT reconstruction and Doppler analysis are achieved in wavenumber domain, researchers often refer to OCT bandwidth in wavelength. Therefore, certain examples provide both wavenumber and wavelength values in the following description. An example OCT source has a square-shaped spectrum such as with a (0.19×10⁵) rad/cm (100-nm) bandwidth and a (1.10×10⁵) rad/cm (570 nm) center wavelength. FIG. 1(a) depicts a selection of Gaussian windows for sub-bands of the OCT spectrum. The Gaussian windows used in STFT have a (0.02×10⁵) rad/cm (9˜12 nm, depending on the wavelength) standard deviation, and the center wavenumbers range from (1.04×10⁵) rad/cm (604 nm) to (1.16×10⁵) rad/cm (542 nm) at 0.03 rad/cm interval, as illustrated in FIG. 1(a), for example. In certain examples, z₀ and z₀+δz are used to represent depth locations of a moving scatter in two sequential OCT A-lines, and δz is an axial displacement. Certain examples analyze seven flow velocities whose axial displacements between two sequential A-lines are 0, δz₀, 2δz₀ . . . 6δz₀, where δz₀ is 35.7 nm. According to Eq. (5), 6δz₀ leads to a 2π Doppler phase shift, assuming that the center wavelength is 570 nm and that refractive index is 1.33. FIG. 1(b) shows example sub-band dependent Doppler phase shifts under different axial displacements, where the dots indicate measured phase shifts and the dashed lines show the fitted linear relationship. Each subset B1, B2, B3, B4, B5 represents one of the OCT sub-bands in FIG. 1(a). As shown in the example of FIG. 1(b), 0 and 6δz₀ displacements would both yield zero flow velocity if measuring Doppler phase shift with the whole spectrum due to phase wrapping. However, the slope of sub-band dependent phase shift array is uniquely related to displacement values, unaffected by phase wrapping. Another interesting result occurs in the case of 3δz₀, where a 2π shift exists in B3 sub-band. This is because the displacement induced phase shifts are so close to π that phase wrapping occurs among different sub-bands. This is referred to as spectroscopic phase shift discontinuity, which is to be corrected before linearly fitting different phase shift values with sub-band center wavenumbers. Although this phase shift discontinuity can also happen when flow induced phase shifts are around 3π, 5π, and etc., washout effect limits the measurable axial flow velocity within twice PLV, corresponding to 2π Doppler phase shift. Therefore, the spectroscopic phase shift discontinuity that happens around π is to be corrected.

Flow Velocity Calculation in Spectroscopic Doppler OCT

In certain examples, Doppler OCT can be used to measure blood flow velocity by averaging Doppler phase shift values of all pixels within a blood vessel cross-section. In cases where the flow velocity exceeds PLV, phase unwrapping is to be implemented in conventional Doppler OCT to obtain a correct measurement result. If the flow velocity is constant throughout the blood vessel, phase unwrapping can be done by adding or subtracting 2π to all the pixels. However, the actual flow profile within the vessel follows an approximate three-dimensional paraboloid. When only the flow velocity in the center of blood vessel exceeds PLV, it is challenging to find the boundary between wrapped and non-wrapped areas. The state-of-art two-dimensional algorithms to correct this type of phase wrapping are usually iterative processes and often generate biased offset error in noisy dataset.

In spectroscopic Doppler OCT, certain examples can retrieve flow velocity based on the linear relationship between phase shifts and center wavenumbers of OCT sub-bands. Phase wrapping only affects the intercept of the fitted line without changing its slope. Since the flow velocity is extracted from the slope of the fitted line, phase unwrapping is not required. However, spectroscopic phase shift discontinuity, as demonstrated in FIG. 1(b), can be corrected.

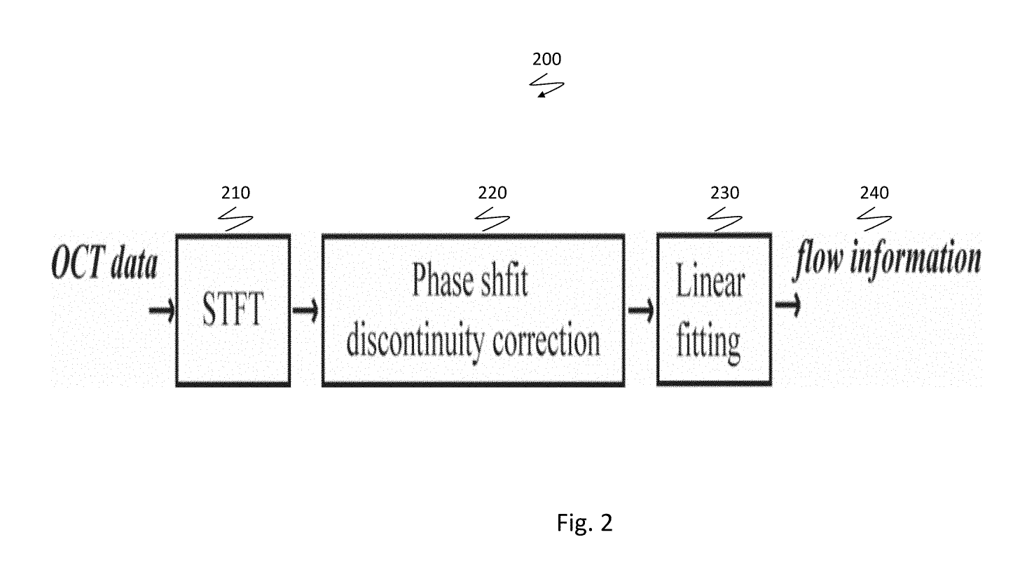

FIG. 2 illustrates a flow diagram of an example process 200 of spectroscopic Doppler analysis. As shown in the example of FIG. 2, at block 210, a short time Fourier transform (STFT) is performed. For example, an STFT is performed with respect to a broadband OCT B-scan to obtain a set of sub-band B-scans. Phase shift values are calculated in each sub-band image using conventional Doppler OCT processing. A region within a target blood vessel is segmented from one sub-band phase shift image, and an obtained boundary is applied to all sub-bands.

At block 220, a correction for phase shift discontinuity is determined. For example, average phase shifts within the blood vessel can be calculated for each sub-band. If there is no spectroscopic phase shift discontinuity, the average phase shift Δ̅φ̅_{D} (τ) can be calculated as follows:

$${\overline{Δϕ}}_{D} ( τ ) = \frac{\sum {Δϕ}_{D} [ τ , ( x , z ) ]}{N} , \qquad ( Eq . 12 )$$

where τ is a center wavenumber of the sub-band used in STFT, N is a number of pixels inside a blood vessel used for spatial averaging, and (x,z) is a coordinate of the corresponding pixel in the B-scan image.

However, if the phase shift values of some pixels are close to π, spectroscopic phase shift discontinuity may occur. To preserve the linear relationship, the average phase shift should be calculated as follow instead:

$${\overline{Δϕ}}_{D} ( τ ) = \frac{\sum {Δϕ}_{D} [ τ , ( x , z ) ]}{N} + \frac{\sum {Δϕ}_{corr} [ τ , ( x , z ) ]}{N} , \qquad ( Eq . 13 )$$

where a correction term is added to compensate for the discontinuity. To calculate the correction term, a phase shift image reconstructed on an arbitrary sub-band τ0 is selected as a reference image, and the reference image is subtracted from phase shift images of all the sub-bands as:

ψ[τ,(x,z)]=|Δφ_{D}[τ,(x,z)]−Δφ_{D}[τ₀,(x,z)]| (Eq. 14).

ψ[τ,(x,z)] is expected to be small if no discontinuity occurs. Therefore, the discontinuity is compensated by adding or subtracting 2π when ψ[τ,(x,z)] exceeds a threshold value:

$${Δϕ}_{corr} [ τ , ( x , z ) ] = \{ \begin{matrix}2 π \cdot sgn \{ {Δϕ}_{D} [ {τ}_{0} , ( x , z ) ] \} , & ψ [ τ , ( x , z ) ] > th , \\ 0 , & ψ [ τ , ( x , z ) ] \leq th ,\end{matrix} , \qquad ( Eq . 15 )$$

where sgn(·) is a sign function. As discussed above, spectroscopic phase shift discontinuity happens when flow induced phase shift is approximately π, and a maximum measurable phase shift should be smaller than 2π due to washout effect. Therefore, a threshold value th can be defined as:

$$th = \frac{2 π ( τ - {τ}_{0} )}{{τ}_{0}} . \qquad ( Eq . 16 )$$

At block 230, linear fitting is performed. For example, a linear regression is performed with respect to sub-band dependent average phase shift values using least-squares fit to extract the slope of the fitted linear relationship:

$$a = \frac{d {\overline{Δϕ}}_{D} ( τ )}{d τ} . \qquad ( Eq . 17 )$$

Then, at block 240, axial flow can be calculated using Eq. (11).

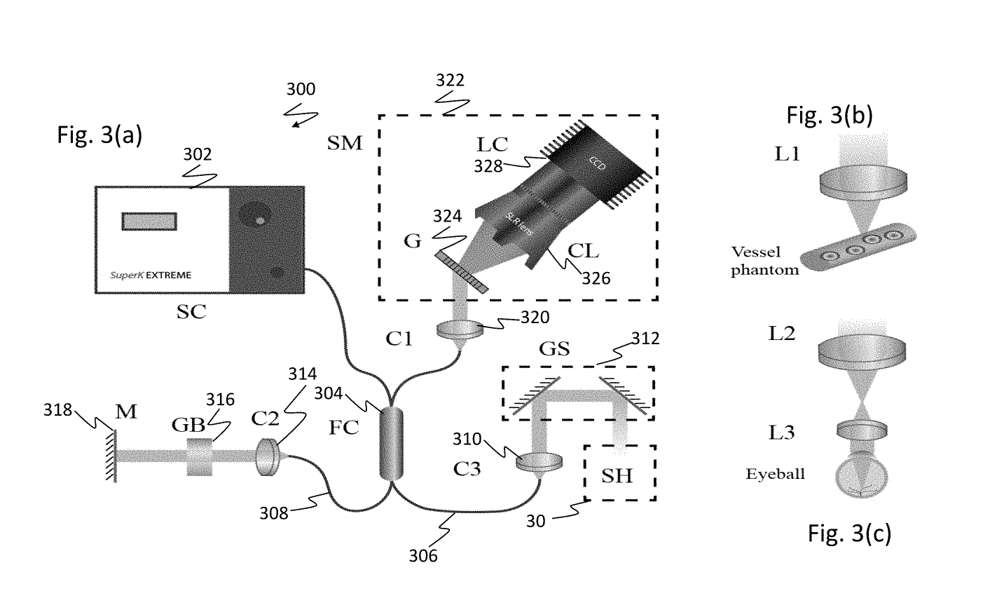

FIG. 3(a) illustrates a schematic of an example spectroscopic Doppler analysis system 300 for vis-OCT blood flow velocity measurement. The example system 300 includes a broadband supercontinuum laser (SC) 302 (e.g., Superk EXTREME, NKT Photonics) to generate visible light source (e.g., a 90-nm bandwidth visible light source centered at 565 nm, which is also used in a clinical setting, etc.). The source beam is coupled into a 2 by 2 fiber coupler 304 (e.g., Nufern 460-HP, 50:50 splitting ratio, GouldFiber Optics), which delivers the light to a sample arm 306 and a reference arm 308. In the sample arm 306, the illumination light is collimated 310 and scanned by a set of galvanometer mirrors 312 (e.g., 6210H, Cambridge Technology). In some examples using a phantom, the illumination beam can be focused using an achromatic doublet (e.g., Edmund Optics) with a focal length of 35 mm (as shown in the example of FIG. 3(b)). In some in vivo examples, a pair of achromatic doublets with focal lengths of 75 mm and 15 mm, respectively, can be used to relay the illumination beam onto the cornea (as shown in the example of FIG. 3(c)). In the reference arm 308, after collimation by a collimator 314, the beam propagates through a glass block 316 (e.g., a BK7 glass block) for dispersion compensation and is reflected by a mirror 318. Backscattered light from the sample arm 306 interferes with back-reflected light from the reference arm 308 in the fiber coupler 304, and an interferogram is collimated by a collimator 320 collected by a spectrometer (SM) 322 including a transmission grating 324 (e.g., 1800 line/mm, Wasatch Photonics), a SLR lens 326 (e.g., 85 mm, f/1.4, Samyang), and a line-scan camera 328 (e.g., spL4096-70 km, Basler), for example. In an example, although the camera 328 includes 4096 linear pixels, only the center 2048 linear pixels are used for spectrum acquisition.

### Example Phantom Validation

In an example, a flow phantom made from 0.5% intralipid (e.g., Sigma, I141-100ML) is imaged in a plastic tube with 125-μm inner diameter. A refractive of intralipid is 1.34, for example. Flow rate is controlled by a motorized syringe pump (e.g., Model A99-EM, Razel Scientific Instruments) within a speed ranging from 0 to 0.25 μL/s. The Doppler angle of the tube is 83.5°. The OCT A-line rate is 20 kHz. 2-mm wide B-scans are acquired crossing the plastic tube, the B-scans including 2048 A-lines. In each measurement, 16 repeated B-scans are performed for averaging. In phantom-based validation of this example, flow rate is used instead of flow velocity because flow rate, including both velocity and cross-sectional area of flow, is of great interest in hemodynamic and metabolic studies. Using flow rate is also more convenient because the true flow rate values can be obtained from the syringe pump without the need to accurately measure the diameter of plastic tube. Therefore, flow velocity provided by Doppler OCT can be converted to flow rate for comparison in the phantom validation study.

Flow induced phase shift can be measured using both conventional and spectroscopic Doppler analyses. In conventional Doppler analysis, averaged phase shift can be obtained inside the tube both with and without phase unwrapping. Before phase unwrapping, the averaged phase shift images can be filtered with a 3×15 (axial×lateral) pixels Doppler filter, since phase unwrapping is sensitive to noise. In this example, an area close to an outer boundary of the flow area on the phase shift B-scan is selected as a reference, where no phase wrapping occurs due to its low flow velocity. Then the flow area is searched, and phase wrapped pixels are labeled if their values changed more than π compared with adjacent pixels. Finally, 2π can be added or subtracted to the labeled pixels to recover the unwrapped phase shift values. In spectroscopic Doppler analysis, 16 Gaussian windows are generated for STFT, whose center wavenumbers evenly distributed from (1.06×105) rad/cm (593-nm) to (1.16×105) rad/cm (542 nm), for example. The windows have a standard deviation of (0.02×105) rad/cm (10 nm at 565-nm wavelength). STFT, corrected for possible spectroscopic phase discontinuity, is applied, and sub-band dependent phase shifts are calculated.

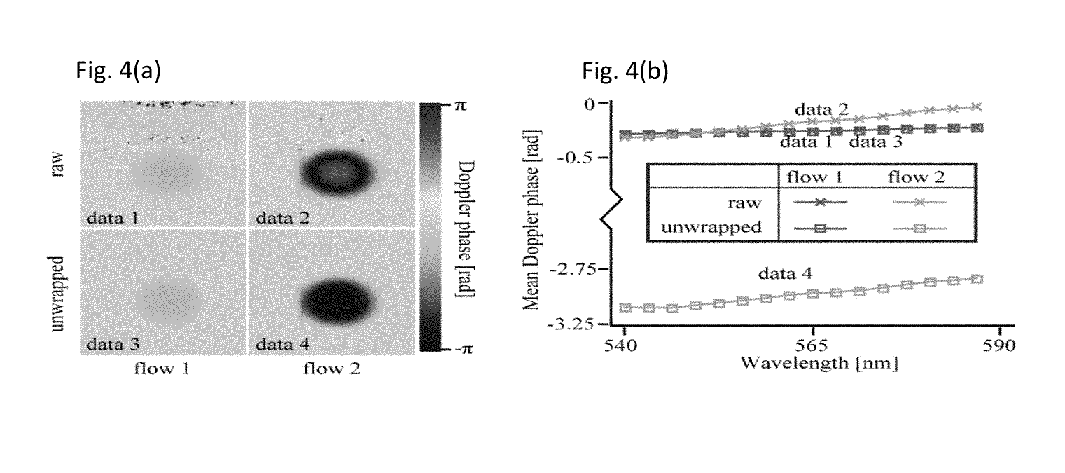

FIG. 4(a) shows example phase shift B-scans under two flow rates before and after phase unwrapping. There is no phase wrapping in Flow 1 (0.021 μL/s) and obvious phase wrapping in Flow 2 (0.218 μL/s). Therefore, before phase unwrapping, the averaged phase shift values throughout flow area are similar between Flow 1 and Flow 2, which can be shown in FIG. 4(b) by the vertical positions of linear functions (Data 1 and Data 2). However, there is a clear difference between the slopes of the two linear functions. After phase unwrapping, the difference in flow rates between Flow 1 and Flow 2 can be better appreciated in FIG. 4(b) from both the vertical positions and the slopes of the linear functions (Data 3 and Data 4). The slopes of linear functions for Data 2 and Data 4 are the same, indicating that spectroscopic Doppler analysis can retrieve flow velocity without phase unwrapping.

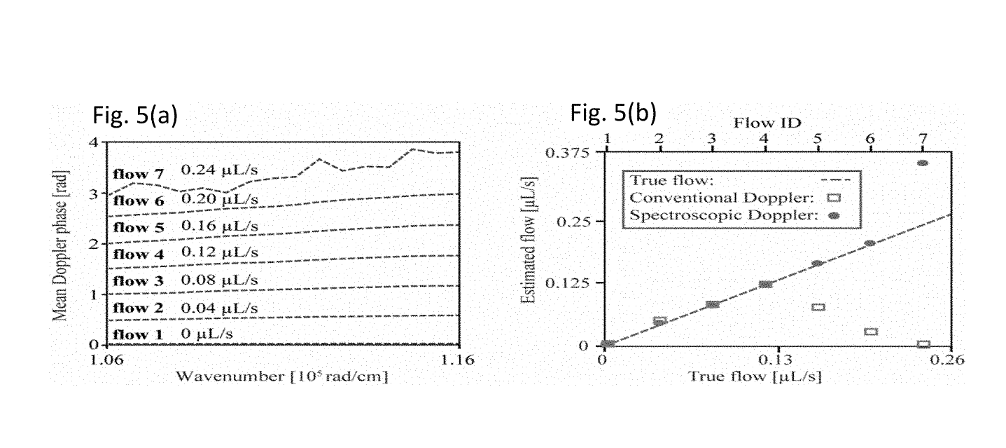

To further compare conventional and spectroscopic Doppler analyses, both methods can be used to measure seven different flow rates in phantom without phase unwrapping, as shown in the example of FIG. 5. To increase flow measurement accuracy, spatial averaging can be applied across the entire flow cross-section in both methods, which provides fair comparison. In this example, the flow rates range from 0 μL/s to 0.24 μL/s with a step size of 0.04 μL/s. In this example, phase wrapping occurs in flow rates greater than 0.12 μL/s. FIG. 5(a) shows sub-band dependent phase shifts at different flow rates. In order to avoid crossing among different phase shift lines and allow better illustration of their slopes, phase shift lines can be manually offset according to the preset flow rates in FIG. 5(a). Therefore, the intercepts of different lines do not represent real calculated values. FIG. 5(b) compares measured flow rates using both methods. Since no phase wrapping exists in the four lower flow rates, both methods can accurately retrieve the preset values. For the three higher rates, conventional Doppler OCT fails to provide accurate measurement results due to phase wrapping. However, spectroscopic Doppler OCT is still able to quantify 0.18 μL/s and 0.21 μL/s. In the case of 0.24 μL/s, severe washout effect occurs in the center of the flow area, which decreases the signal-to-noise ratio (SNR). Therefore, the sub-band dependent phase shift array is unstable, whose fitted slope value failed to provide an accurate estimate for the flow rate.

### Example In Vivo Retinal Blood Flow Measurement

In certain examples, both conventional and spectroscopic Doppler analyses can be used to compare measured retinal blood flow velocity without phase unwrapping (Long Evans, 300 g, Charles River) in vivo (e.g., using Long Evans, 300 g, Charles River rats, etc.). In this example, vis-OCT measurements are conducted in rat eyes using a circular scanning protocol. The diameter of scanning circle in this example is approximately 0.5 mm on the rat retina. Each circular scan includes 4096 A-lines. The vis-OCT probing power is 0.8 mW on the cornea, for example. First, conventional Doppler analysis is used to calculate phase shift images using the whole broadband spectrum. Then, 24 vessels are randomly selected with phase shifts roughly distributed between 0.2 rad and 1 rad. Spectroscopic analysis is applied to these selected vessels, using the same set of Gaussian windows as in the phantom experiment but adjusting the standard deviation to 0.03×105 rad/cm (around 15 nm at 565-nm wavelength). STFT is applied to the raw OCT interferograms to obtain a series of phase shift images. Flow areas can be manually segmented within retinal blood vessels from sub-band phase shift images. Axial flow velocities are obtained, converted to phase shift values, and compared with the results from conventional Doppler method.

In this example, axial flow velocity, rather than flow rate, can be used to compare conventional and spectroscopic Doppler methods. In in vivo studies, flow rates across different vessels may be similar, while variation in the Doppler angles among different blood vessels produces distinct axial flow velocities. Therefore, by using axial flow velocity without calculating flow rate, performance of conventional and spectroscopic Doppler methods at a wide range of phase shift values.

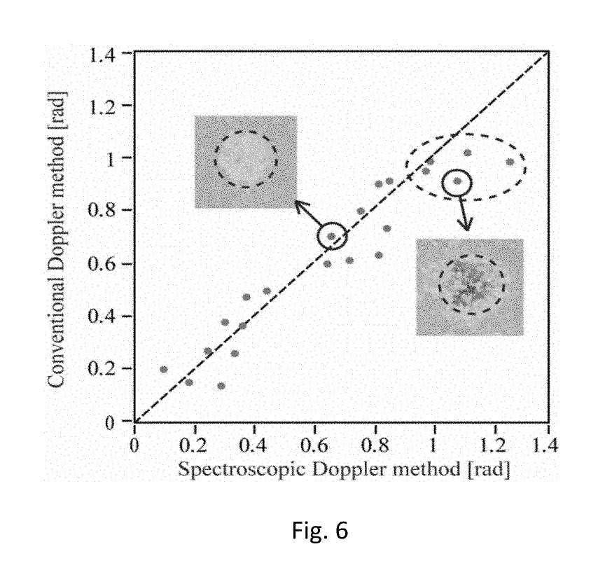

FIG. 6 compares example rat retinal axial flow velocities represented by Doppler phase shifts using conventional and spectroscopic Doppler analyses without phase unwrapping. The scatterplot shows a good correlation between phase shifts measured by two methods when mean Doppler phase shift is smaller than 1 rad. However, there is a larger variation as compared with the phantom results shown in FIGS. 5(a)-5(b) due to reduced SNR. The example of FIG. 6 also shows that the five highlighted data points all lie below the diagonal line, suggesting lower flow velocity estimation by the conventional Doppler analysis. This is consistent with the phantom experiment in that conventional Doppler OCT method tends to underestimate flow velocity when phase wrapping occurs. The dashed line in FIG. 6 suggests identical values measured by both methods. The dashed ellipse highlights five cases where phase wrapping possibly occurs. The two insets are cross-sectional phase images of two selected blood vessels.

### Summary of Examples

Thus, certain examples provide a spectroscopic Doppler analysis for flow velocity measurement. Rather than calculating Doppler phase shift using a broadband OCT interferogram, as in conventional Doppler OCT, STFT is used to reconstruct a series of complex OCT B-scans in different sub-bands, whose Doppler phase shifts were then calculated separately. Rather than averaging the phase shift values for improved SNR, the phase shift values are fit against the center wavenumbers of the sub-bands using a linear function, whose slope is related to the axial flow velocity. In certain examples, spectroscopic Doppler analysis is unaffected by phase wrapping since the absolute shift values are not directly used in velocity calculation. Therefore, spectroscopic Doppler analysis does not rely on phase unwrapping to measure vascular flow whose center velocity exceeds PLV. Using sub-bands with reduced bandwidth decreases the axial resolution in the Doppler phase shift images, which affects measurement accuracy if the flow area cannot be clearly resolved at the reduced axial resolution.

Compared with conventional Doppler OCT and its phase unwrapping techniques, spectroscopic Doppler analysis is a good alternative for flow velocity measurement in vis-OCT. Working on shorter wavelengths, vis-OCT has much improved axial resolution but is more susceptible to phase wrapping. Due to higher resolution, the phase shift images of vis-OCT retain sufficient information even only reconstructed on a spectral sub-band. Due to much lower PLV, vis-OCT requires more careful phase unwrapping procedures when measuring flow velocity than using conventional Doppler OCT method. Though phase unwrapping can provide satisfactory results when image SNR is guaranteed, it is not trivial, and even biased if the image quality is moderate, especially in clinical settings. In addition, the unwrapping errors may accumulate during the iterative process adopted in most unwrapping algorithms. Therefore, spectroscopic analysis is especially helpful in vis-OCT, though it can also be applied to conventional near-infrared (NIR) OCT as well.

The total number and bandwidth of the spectral sub-bands affect flow measurement accuracy. Limited number of sub-bands leads to fewer points for linear regression, which makes the fitted slope susceptible to noise. Too many sub-bands, on the other hand, involve more intensive computation with marginal benefit. In certain examples, 16 sub-bands are selected to perform spectroscopic analysis, which is a tradeoff between accuracy and computation efforts. Selection of the sub-bands can be confirmed using numerical simulation. Selecting proper bandwidth for sub-bands is also important. A large bandwidth decreases the spectral fitting range and a small bandwidth leads to not only reduced resolution but also lower SNR. In an example, (0.04×105) rad/cm is used for phantom experimental verification and (0.06×105) rad/cm is used for animal experimental verification. (Note that the sub-band width is twice the standard deviation of Gaussian window used in STFT.) A larger bandwidth is used in animal experiment to limit the phase noise, since the SNR of in vivo data is naturally lower than phantom data.

Though spectroscopic Doppler analysis is robust to phase wrapping, it is still limited by fringe washout effect in spectrometer-based SD-OCT. The sub-band dependent phase shift becomes noisy when significant washout occurs and the slope extracted from linear fitting fails to reveal the flow velocity. SS-OCT is almost unaffected by fringe washout effect since it increases washout threshold by a factor that equals the total number of spectral samples in each A-line. Therefore, in certain examples, spectroscopic Doppler analysis can extend the velocity measurement range in SS-OCTs.

In certain examples, sufficient SNR is important to obtain reliable flow velocity using spectroscopic Doppler analysis. To help guarantee sufficient SNR, spatial averaging can be applied across the entire flow cross-section in both phantom and animal studies. Since the flow velocity is translated from slope of the fitted straight line, accurate measurement involves consistency of phase shift values calculated from spectral sub-bands, which themselves can be more susceptible to influence from noise due to reduced spectral bandwidth.

Thus, certain examples provide a spectroscopic Doppler analysis for flow velocity measurement in vis-OCT, tested and verified in both phantom and in vivo experiments. The spectroscopic Doppler analysis method uses spectroscopic information of each phase-shifted image pixel to retrieve flow velocity unambiguously, rather than depending solely upon the pixel spatial relationship to correct the phase wrapping as in conventional Doppler OCT method. SS-OCT can benefit from this Doppler analysis method as well as spectrometer-based OCT.

### IV. Example Software and Computer Systems

In various examples, the methods and systems of the present disclosure may further include software programs on computer systems and use thereof. Accordingly, computerized control for the synchronization of system functions such as laser system operation, fluid control function, and/or data acquisition steps are within the bounds of the invention. The computer systems may be programmed to control the timing and coordination of delivery of sample to a detection system, and to control mechanisms for diverting selected samples into a different flow path. In some examples, the computer may also be programmed to store the data received from a detection system and/or process the data for subsequent analysis and display.

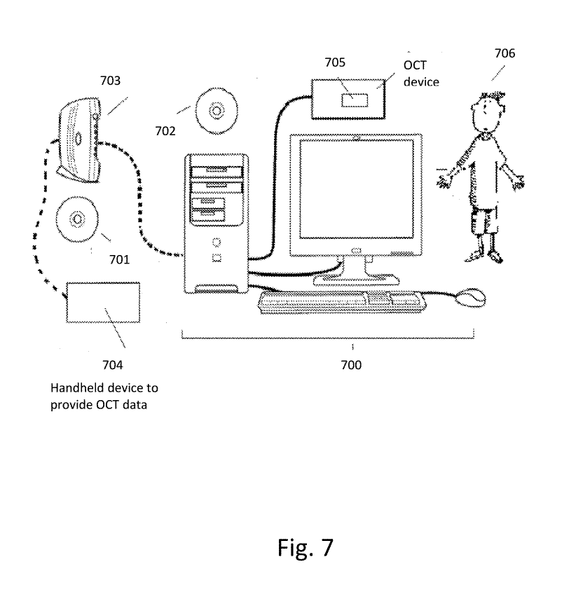

The computer system 700 illustrated in FIG. 7 may be understood as a logical apparatus that can read instructions from media 702 and/or a network port, which can optionally be connected to server 703 having fixed media 702. The system, such as shown in FIG. 7 can include a CPU, disk drives, optional input devices such as handheld devices for acquiring flow measurement data 704 or other instrument types such as a laboratory or hospital based instrument 705. Data communication can be achieved through the indicated communication medium to a server at a local or a remote location. The communication medium can include any suitable device for transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 706 as illustrated in FIG. 7.

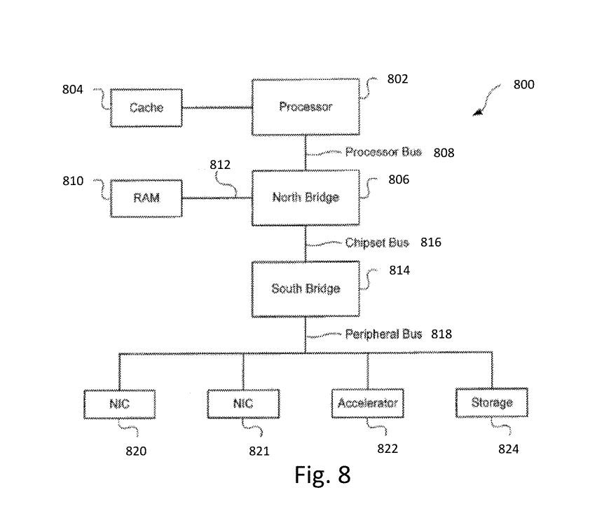

FIG. 800 is a block diagram illustrating a first example architecture of a computer system 800 that can be used in connection with the present disclosure. As depicted in FIG. 8, the example computer system can include a processor 802 for processing instructions. Non-limiting examples of processors include: Intel Xeon™ processor, AMD Opteron™ processor, Samsung 32-bit RISC ARM 1176JZ(F)-S vl.O™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some examples, multiple processors or processors with multiple cores can also be used, whether in a single computer system, in a cluster, or distributed across systems over a network comprising a plurality of computers, cell phones, and/or personal data assistant devices.

As illustrated in FIG. 8, a high speed cache 804 can be connected to, or incorporated in, the processor 802 to provide a high speed memory for instructions or data that have been recently, or are frequently, used by processor 802. The processor 802 is connected to a north bridge 806 by a processor bus 808. The north bridge 806 is connected to random access memory (RAM) 810 by a memory bus 812 and manages access to the RAM 810 by the processor 802. The north bridge 806 is also connected to a south bridge 814 by a chipset bus 816. The south bridge 814 is, in turn, connected to a peripheral bus 818. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 818. In some alternative architectures, the functionality of the north bridge can be incorporated into the processor instead of using a separate north bridge chip.

In some examples, system 800 can include an accelerator card 822 attached to the peripheral bus 818. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 824 and can be loaded into RAM 810 and/or cache 804 for use by the processor. The system 800 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with the present disclosure.

In this example, system 800 also includes network interface cards (NICs) 820 and 821 connected to the peripheral bus for providing network interfaces to external storage, such as Network Attached Storage (NAS) and other computer systems that can be used for distributed parallel processing.

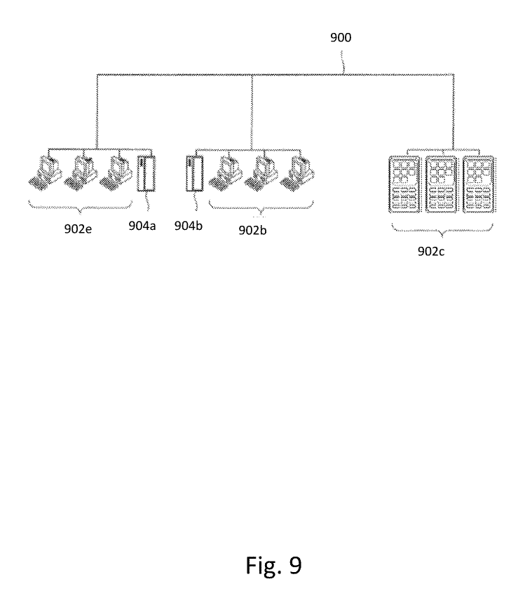

FIG. 9 is a diagram showing a network 900 with a plurality of computer systems 902a, and 902b, a plurality of cell phones and personal data assistants 902c, and Network Attached Storage (NAS) 904a, and 904b. In some examples, systems 902a, 902b, and 902e can manage data storage and optimize data access for data stored in Network Attached Storage (NAS) 904a and 904b. A mathematical model can be used for the data and be evaluated using distributed parallel processing across computer systems 902a, and 902b, and cell phone and personal data assistant systems 902c. Computer systems 902a, and 902b, and cell phone and personal data assistant systems 902c can also provide parallel processing for adaptive data restructuring of the data stored in Network Attached Storage (NAS) 904a and 904b. FIG. 9 illustrates an example only, and a wide variety of other computer architectures and systems can be used in conjunction with the various examples of the present invention. For example, a blade server can be used to provide parallel processing. Processor blades can be connected through a back plane to provide parallel processing. Storage can also be connected to the back plane or as Network Attached Storage (NAS) through a separate network interface.

In some examples, processors can maintain separate memory spaces and transmit data through network interfaces, back plane or other connectors for parallel processing by other processors. In other examples, some or all of the processors can use a shared virtual address memory space.

The above computer architectures and systems are examples only, and a wide variety of other computer, cell phone, and personal data assistant architectures and systems can be used in connection with example examples, including systems using any combination of general processors, co-processors, FPGAs and other programmable logic devices, system on chips (SOCs), application specific integrated circuits (ASICs), and other processing and logic elements. In some examples, all or part of the computer system can be implemented in software or hardware. Any variety of data storage media can be used in connection with example examples, including random access memory, hard drives, flash memory, tape drives, disk arrays, Network Attached Storage (NAS) and other local or distributed data storage devices and systems.

In some examples, the computer system can be implemented using software modules executing on any of the above or other computer architectures and systems. In other examples, the functions of the system can be implemented partially or completely in firmware, programmable logic devices such as field programmable gate arrays, system on chips (SOCs), application specific integrated circuits (ASICs), or other processing and logic elements. For example, the Set Processor and Optimizer can be implemented with hardware acceleration through the use of a hardware accelerator card, such as accelerator card.

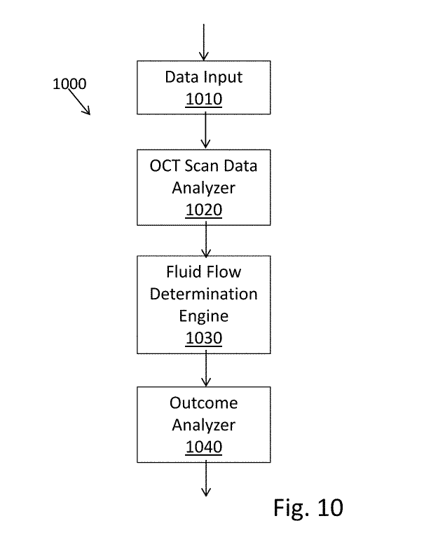

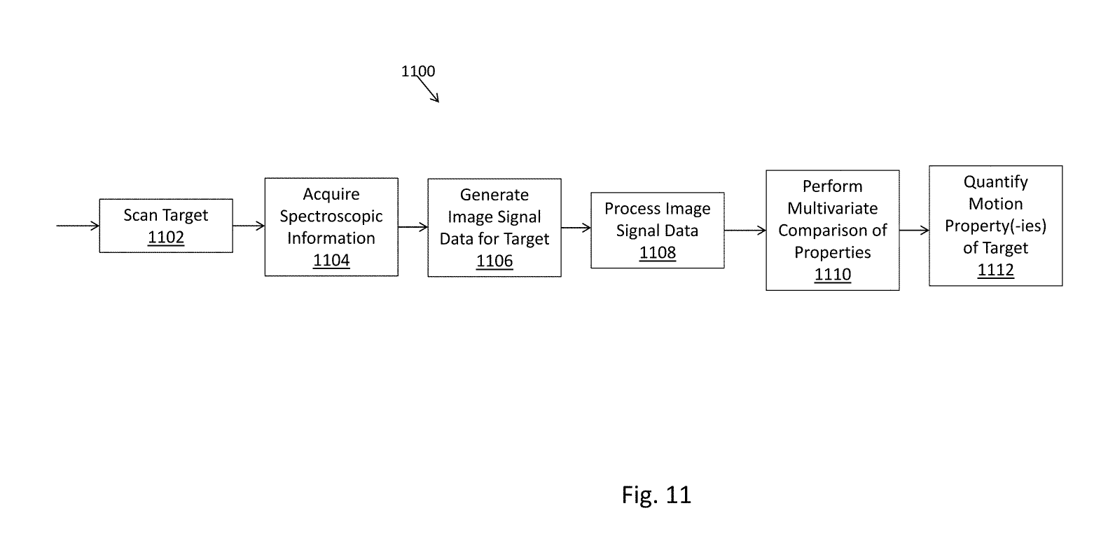

For example, as shown in FIG. 10, an OCT data processing system 1000 includes a data input 1010 to receive OCT scan data from scanning of a target by an OCT device. One or more OCT scans may be generated and obtained by one or more components of the OCT device/system. The example system 1000 includes an OCT scan data analyzer 1020 to process/analyze the OCT scan data according to one or more criterion. The example system 1000 includes a fluid flow determination engine 1030 to determine fluid flow in the target based on the analysis from the data analyzer 1020. Fluid flow information can be provided by the engine 1030 to an outcome analyzer 1040 to provide feedback and/or other output (e.g., display of information, printout of information, relay of information to another system (e.g., to drive another process), etc.). For example, the outcome analyzer 1040 can provide information to a healthcare practitioner and/or diagnostic system to facilitate a medical decision.

The example system 1000 can be used to execute an example method 1100 to evaluate a target. At block 1102, a target (e.g., tissue, blood cells, etc.) is scanned with one or more beams of incident low coherence radiation. For example, the low coherence radiation is selected from one or more regions of the electromagnetic spectrum based on the target selected (e.g., a single beam of low coherence light in the visible light range, etc.).

At block 1104, spectroscopic information is acquired from scanning signals generated by the interaction of the incident low coherence radiation and the target. At block 1106, image signal data is generated for the target from the scanning signals.

At block 1108, the image signal data is processed by selecting one or more electro-magnetic properties related to the modulation of the low coherence radiation by variation of relative permittivity and conductivity of the target. For example, processing of the image signal data includes splitting broad spectrum signal into one or more narrower sub-bands for performing a multivariate comparison. A width and quantity of sub-bands used to split the broad spectrum signal is determined by the target selected, by selected electro-magnetic properties, by the quantifying one or more motion properties of the target, etc. In other examples, processing of the image signal data includes obtaining one or more Doppler phase shifts from one or more narrower sub-bands.

At block 1110, a multivariate comparison of the selected electro-magnetic properties related to the modulation of the low coherence radiation is performed by variation of relative permittivity and conductivity of the target. For example, performing a multivariate comparison can include a linear fitting of the one or more Doppler phase shifts with respect to center wavelengths of the one or more sub-bands and determining a slope. The multivariate comparison can include a Fourier-related transform of image signal data, for example. The multivariate comparison can include compensation for phase shift discontinuity, for example.

At block 1112, one or more motion properties of the target are quantified. A motion property of the target includes flow velocity (e.g., blood flow velocity), etc. In certain examples, the flow velocity is used to determine metabolic activity of the target. In certain examples, the target is tissue or one or more red blood cells. In certain examples, the electro-magnetic property related to the modulation of the low coherence radiation by variation of relative permittivity and conductivity of the target is the absorption of visible light of hemoglobin in the target. In certain examples, quantifying one or more motion properties of the target is an average of one or more measurements. In certain examples, quantifying one or more motion properties of the target does not rely on phase unwrapping. In certain examples, quantifying one or more motion properties of the target is used to make a medical decision. In certain examples, quantifying one or more motion properties of the target is used in combination with one or more measurements of oxygen concentration to determine metabolic activity. In certain examples, quantifying one or more motion properties of the target and one or more measurements of oxygen concentration is determined from a single scan of the target.

### V. Example Terminology

The terminology used therein is for the purpose of describing particular examples only and is not intended to be limiting of a device of this disclosure. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Several aspects of a device of this disclosure are described above with reference to example applications for illustration. It should be understood that numerous specific details, relationships, and methods are set forth to provide a full understanding of a device. One having ordinary skill in the relevant art, however, will readily recognize that a device can be practiced without one or more of the specific details or with other methods. This disclosure is not limited by the illustrated ordering of acts or events, as some acts may occur in different orders and/or concurrently with other acts or events. Furthermore, not all illustrated acts or events are required to implement a methodology in accordance with this disclosure.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another example. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5.

### VI. Brief Description of Certain Examples

Optical coherence tomography (OCT) is a routine imaging modality in eye examination. In addition to providing three-dimensional structure imaging, a functional extension of OCT is to measure flow velocity in the blood vessel using Doppler phase shift. Certain examples provide a new methodology to measure blood flow, which overcomes challenges in existing system and extends the measurement range of blood flow. This new method provides higher measurement accuracy than the start-of-the-art, does not require additional hardware modification, and can be implemented to all OCT systems, including spectrometer based spectral-domain OCT and swept-source OCT.

Certain examples can be used in diagnosing and managing several diagnosis diseases, including, for example, glaucoma, retinal diabetic retinopathy, and age-related macular degeneration.

In contrast to other techniques, certain examples do not need spatial information to determine phase wrapping region. Certain examples can measure flow velocity unambiguously. Certain examples are robust to multiple orders of phase wrapping. Certain examples extend the measurement range of blood flow.

Certain examples provide spectroscopic analysis for Doppler optical coherence tomography (OCT). In certain examples, the broadband OCT spectrum is divided into a series of narrow sub-bands, and OCT images are reconstructed to extract corresponding Doppler phase shifts in all the sub-bands. Then, sub-band dependent phase shift is regressed against a linear function. The slope of the fitted straight line, unaffected by phase wrapping, is used to quantity flow velocity while the intercept is disinterested.

Thus, the problem of phase wrapping can be solved in Doppler OCT when measuring high flow velocity. An accurate blood flow measurement range can be achieved, for example, without troublesome phase unwrapping.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed:

1. A method of evaluating a target, the method comprising:
a. performing scanning of a target with one or more beams of incident low coherence radiation, wherein the low coherence radiation is selected from one or more regions of the electromagnetic spectrum based on the target selected;
b. acquiring spectroscopic information from scanning signals generated by the interaction of the incident low coherence radiation and the target;
c. generating image signal data for the target from the scanning signals;
d. processing the image signal data by selecting one or more electro-magnetic properties related to the modulation of the low coherence radiation by variation of relative permittivity and conductivity of the target;
e. performing a multivariate comparison of the selected electro-magnetic properties related to the modulation of the low coherence radiation by variation of relative permittivity and conductivity of the target; and
f. quantifying one or more motion properties of the target.

2. The method of claim 1, wherein the scanning of a target with one or more beams of incident low coherence radiation is performed with a single beam of low coherence light in the visible light range.

3. The method of claim 1, wherein the processing of image signal data includes splitting broad spectrum signal into one or more narrower sub-bands to perform a multivariate comparison.

4. The method of claim 3, wherein the width and quantity of sub-bands used to split the broad spectrum signal is determined by the target selected.

5. The method of claim 3, wherein the width and quantity of sub-bands used to split the broad spectrum signal is determined by selected electro-magnetic properties.

6. The method of claim 3, wherein the width and quantity of sub-bands used to split the broad spectrum signal is determined by quantifying one or more motion properties of the target.

7. The method of claim 3, wherein the processing of image signal data includes obtaining one or more Doppler phase shifts from the one or more narrower sub-bands.

8. The method of claim 1, wherein the performing a multivariate comparison includes linear fitting of the one or more Doppler phase shifts with respect to center wavelengths of the one or more sub-bands and determining a slope.

9. The method of claim 1, wherein the performing a multivariate comparison includes a Fourier-related transform of image signal data.

10. The method of claim 1, wherein the performing the multivariate comparison includes compensation for phase shift discontinuity.

11. The method of claim 1, wherein the motion property of the target is flow velocity.

12. The method of claim 12, wherein the flow velocity is used to determine metabolic activity of the target.

13. The method of claim 1, wherein the target is tissue or one or more red blood cells.

14. The method of claim 1, wherein the electro-magnetic properties related to the modulation of the low coherence radiation by variation of relative permittivity and conductivity of the target is the absorption of visible light of hemoglobin in the target.

15. The method of claim 1, wherein the quantifying one or more motion properties of the target is an average of one or more measurements.

16. The method of claim 1, wherein the quantifying one or more motion properties of the target does not rely on phase unwrapping.

17. The method of claim 1, wherein the quantifying one or more motion properties of the target is used to make a medical decision.

18. The method of claim 1, wherein the quantifying one or more motion properties of the target is used in combination with one or more measurements of oxygen concentration to determine metabolic activity.

19. The method of claim 19, wherein the quantifying one or more motion properties of the target and one or more measurements of oxygen concentration is determined from a single scan of the target.

20. A system comprising:
a memory to store instructions; and
a processor to execute the instructions to at least:
a. perform scanning of a target with one or more beams of incident low coherence radiation, wherein the low coherence radiation is selected from one or more regions of the electromagnetic spectrum based on the target selected;
b. acquire spectroscopic information from scanning signals generated by the interaction of the incident low coherence radiation and the target;
c. generate image signal data for the target from the scanning signals;
d. process the image signal data by selecting one or more electro-magnetic properties related to the modulation of the low coherence radiation by variation of relative permittivity and conductivity of the target;
e. perform a multivariate comparison of the selected electro-magnetic properties related to the modulation of the low coherence radiation by variation of relative permittivity and conductivity of the target; and
f. quantify one or more motion properties of the target.
